(19) United States
(12) Patent Application Publication
(10) Pub. No.: US 2023/0152328 A1
(43) Pub. Date: May 18, 2023

(54) LIGHT-EMITTING MARKER

(71) Applicant: Sumitomo Chemical Co., Ltd., Tokyo (JP)

(72) Inventors: Sheena Zuberi, Godmanchester (GB); Nazrul Islam, Godmanchester (GB); Jonathan Behrendt, Godmanchester (GB)

(73) Assignee: Sumitomo Chemical Co., Ltd., Tokyo (JP)

(21) Appl. No.: 17/916,345

(22) PCT Filed: Mar. 30, 2021
(86) PCT No.: PCT/EP2021/058382

§ 371 Date: Sep. 30, 2022

(30) Foreign Application Priority Data
Mar. 31, 2020 (GB) 2004719.7

(51) Int. Cl. G01N 33/58; C09K 11/06; C08G 61/12

(52) U.S. Cl. CPC G01N 33/582; C09K 11/06; C08G 61/126

(57) ABSTRACT

A target analyte in a sample may be identified using a light-emitting ting marker is irradiated and emission from the sample at an emission wavelength of the light-emitting marker is detected. The method may be flow cytometry. The light-emitting marker comprises a light-emitting polymer comprising an electron-accepting repeat unit of formula (I) and an electron-donating repeat unit: (I) R¹ and R² independently in each occurrence is H or a substituent and X in each occurrence is dependently a substituent. The light-emitting marker may be a particulate light-emitting marker comprising particles containing the light-emitting polymer and a matrix material, e.g. silica.

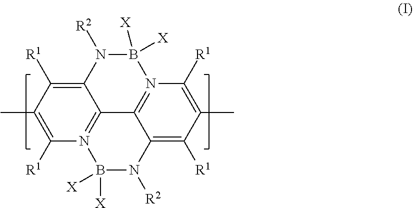

### BACKGROUND

Embodiments of the present disclosure relate to light-emitting markers. Embodiments of the present disclosure further relate to methods of identifying a target analyte using said light-emitting marker.

Light-emitting polymers have been disclosed for marking a target analyte.

WO 2018/060722 discloses composite particles comprising a mixture of silica and a light-emitting polymer having polar groups.

CN105542131B discloses a photovoltaic cell containing a polymer having a structure of formula:

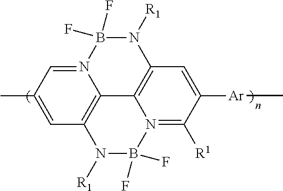

CN110229316A discloses a polymer having a structure of the formula for use an organic solar cell or an organic field effect transistor:

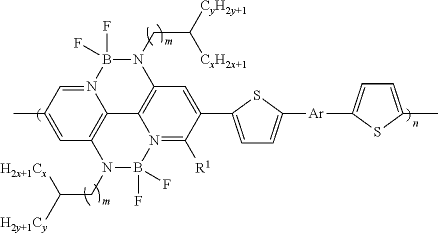

Xiaojing Long et al, “Low-bandgap polymer electron acceptors based on double B→N bridged bipyridine (BNBP) and diketopyrrolopyrrole (DPP) units for all-polymer solar cells”, Journal of Materials Chemistry C, 4 (42), 9961-9967, 2016 discloses a polymer for use in an all-polymer solar cell.

### SUMMARY

In some embodiments, the present disclosure provides a method of identifying a target analyte in a sample to which has been added a first light-emitting marker configured to bind to the target analyte. The sample is irradiated and emission from the light-emitting marker is detected. The light-emitting marker comprises a light-emitting polymer comprising a repeat unit of formula (I):

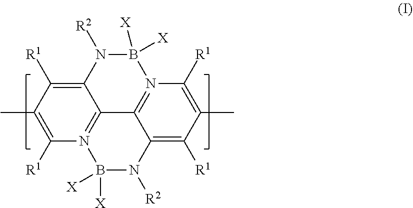

wherein R¹ independently in each occurrence is H or a substituent; R² independently in each occurrence is H or a substituent; and X in each occurrence is independently a substituent.

In some embodiments, the polymer is a homopolymer.

In some embodiments, the polymer is a copolymer comprising repeat units of formula (I) and one or more further repeat units. Optionally, the copolymer comprises an electron-accepting repeat unit of formula (I) and an electron-donating repeat unit. Optionally, the electron donating repeat unit is selected from formulae (IVa)-(IVp):

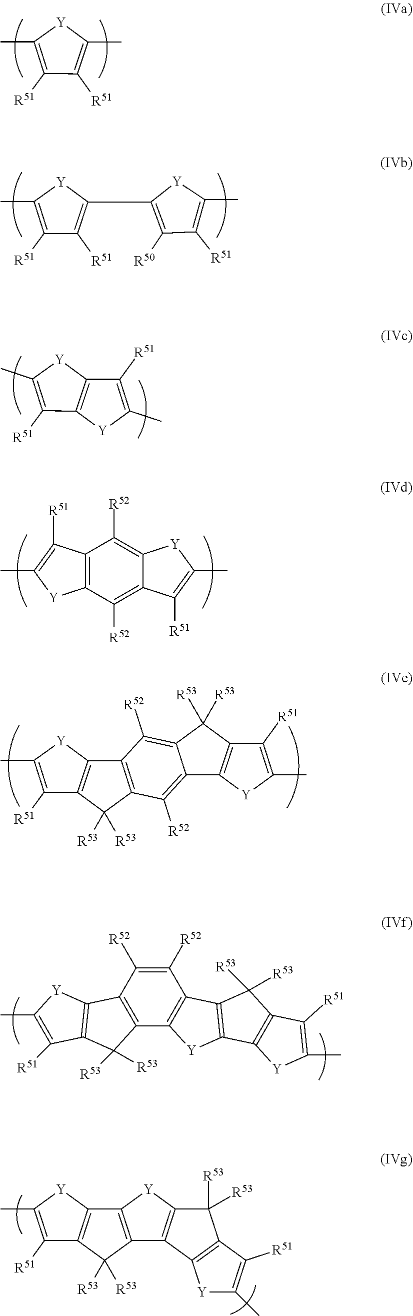

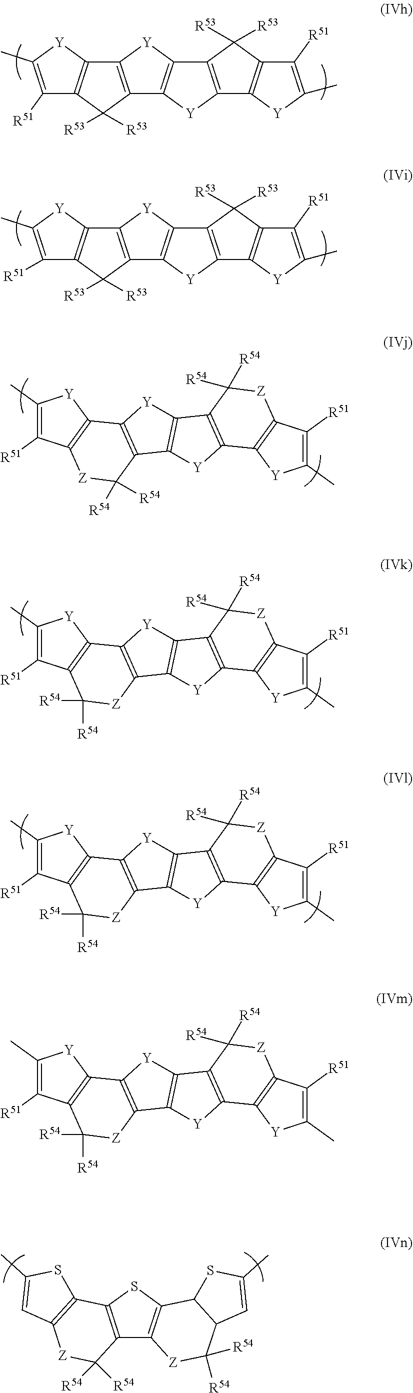

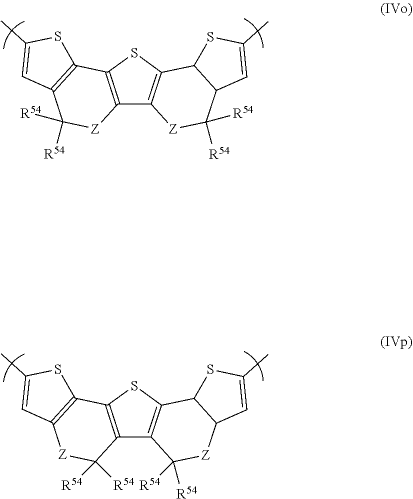

wherein Y in each occurrence is independently O or S; Z in each occurrence is O, NR⁵⁵, or C(R⁵⁴)₂; and R⁵⁰, R⁵¹, R⁵², R⁵³, R⁵⁴ and R⁵⁵ independently in each occurrence is H or a substituent and wherein R⁵⁰ groups may be linked to form a ring.

Optionally, the light-emitting marker comprises a biomolecule configured to bind to the target analyte.

Optionally, the polymer has a solubility in water or a C₁₋₆ alcohol of at least 0.1 mg/ml.

Optionally, at least one of the repeat units of the light-emitting polymer is substituted with at least one ionic substituent.

Optionally, at least one of the repeat units of the light-emitting polymer is substituted with at least one group of formula (III):

—O(R⁴O)ᵥ—R⁵ (III)

wherein R⁴ in each occurrence is a C₁₋₁₀ alkylene group wherein one or more non-adjacent, non-terminal C atoms of the alkylene group may be replaced with O, R⁵ is H or C₁₋₅ alkyl, and v is 0 or a positive integer.

Optionally, the light-emitting polymer has an absorption peak of greater than 550 nm.

In some embodiments, the light-emitting marker is a particulate marker. Optionally, the particles comprise the light-emitting polymer and an inorganic matrix. Optionally, a biomolecule configured to bind to the target analyte is bound to the inorganic matrix.

In some embodiments, the light-emitting marker is dissolved in the sample.

Optionally, the sample comprises one or more additional light-emitting markers different from the first light-emitting marker.

Optionally, each of the one or more additional light-emitting markers comprises a light-emitting material which emits light having an emission peak which is different from that of the light-emitting polymer.

Optionally, the light-emitting material of each additional light-emitting marker has an absorption peak which is different from that of the first light-emitting marker.

Optionally, the first light-emitting marker has an absorption peak having a full width at half maximum (FWHM) which is separated by at least 100 nm from the FWHM of an absorption peak of each of the additional light-emitting markers.

Optionally, the method is a flow cytometry method and the target analyte is a target cell.

In some embodiments, the present disclosure provides a light-emitting marker precursor comprising a light-emitting polymer and a functional group wherein the light-emitting polymer comprises a repeat unit of formula (I):

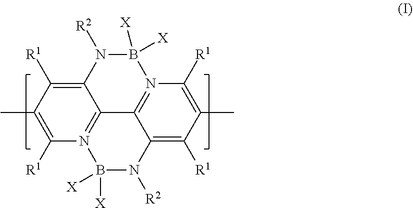

wherein R¹ independently in each occurrence is H or a substituent; R² independently in each occurrence is H or a substituent; and X in each occurrence is independently a substituent. The polymer comprising the repeat unit of formula (I) may be as described anywhere herein.

Optionally, the functional group is biotin.

Optionally, the light-emitting precursor is in particulate form.

In some embodiments, the present disclosure provides a formulation comprising the light-emitting marker precursor dissolved or dispersed in one or more solvents.

In some embodiments, the present disclosure provides a light-emitting marker comprising a light-emitting polymer and a binding group comprising a biomolecule wherein the light-emitting polymer comprises a repeat unit of formula (I):

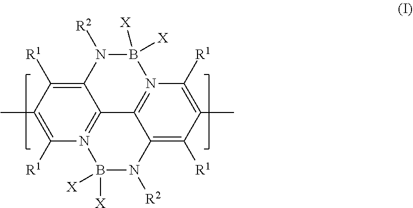

wherein R¹ independently in each occurrence is H or a substituent; R² independently in each occurrence is H or a substituent; and X in each occurrence is independently a substituent. The polymer comprising the repeat unit of formula (I) may be as described anywhere herein.

In some embodiments, the present disclosure provides solution comprising the light-emitting marker dissolved in a solvent.

In some embodiments, the present disclosure provides a method of forming the light-emitting marker, the method comprising reacting the light-emitting marker precursor with a material for forming the binding group.

In some embodiments, the present disclosure provides a light-emitting particle comprising a light-emitting polymer comprising a repeat unit of formula (I):

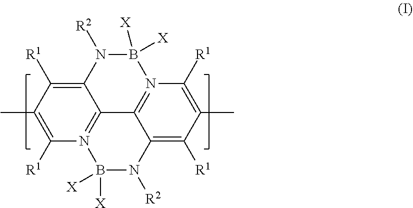

wherein R¹ independently in each occurrence is H or a substituent; R² independently in each occurrence is H or a substituent; and X in each occurrence is independently a substituent. The polymer comprising the repeat unit of formula (I) may be as described anywhere herein.

Optionally, the light-emitting particle comprises the light-emitting material mixed with or covalently bound to a matrix material.

Optionally, the matrix material is an inorganic oxide.

Optionally, the inorganic oxide is silica.

Optionally, the light-emitting particle comprises a core and a shell and wherein the light-emitting material is disposed in at least one of the core and the shell.

Optionally, a functional group is bound to a surface of the light-emitting particle.

Optionally, the functional group is biotin.

In some embodiments, the present disclosure provides a formulation comprising light-emitting particles as described herein dispersed in one or more solvents.

In some embodiments, the present disclosure provides a method of forming the light-emitting particles described herein wherein a monomer for forming silica is polymerised in the presence of the light-emitting material.

### DESCRIPTION OF THE DRAWINGS

The disclosed technology and accompanying figures describe some implementations of the disclosed technology.

FIG. 1 illustrates a flow cytometer for use in a method according to some embodiments;

FIG. 2 shows absorption spectra in methanol solution of polymers according to embodiments of the present disclosure; and

FIG. 3 shows absorption spectra in water of a dispersion of particles containing polymers according to embodiments of the present disclosure.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to an atom include any isotope of that atom unless stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The present inventors have identified certain light-emitting polymers suitable for use in light-emitting markers for detection of a target analyte, such as detection of cells in flow cytometry. The polymers may have a narrow absorption band, which may allow for absorption by light from a light source having an excitation wavelength at or close to an absorption peak of the polymer with little or no absorption from any other light sources having a different excitation wavelength, for example a light source intended for excitation of a light-emitting marker other than that containing the light-emitting polymer. Thus, the light-emitting marker described herein (the “first light-emitting marker”) may be used in combination with one or more other light-emitting markers which contain a light-emitting material which is different from the light-emitting polymer of the first light-emitting markers and which absorb light at a wavelength different from the first light-emitting marker.

A narrow absorption band of the light-emitting polymers described herein may reduce or eliminate the need for compensation due to “cross-talk” arising from absorption by the light-emitting polymer of light from a light source intended for excitation of the light-emitting material of a different light-emitting marker.

The light-emitting marker has a binding group for binding to a target analyte. Preferably, the binding group is a biomolecule binding group.

In some embodiments, the binding group is bound, preferably covalently bound, to the light-emitting polymer. The binding group may be provided as a side group of a repeat unit of the light-emitting polymer or as an end-group of the light-emitting polymer. In use, e.g. during flow cytometry, the light-emitting polymer may be dissolved or dispersed in a sample to be analysed. The light-emitting polymer is preferably dissolved in water.

In some embodiments, the light-emitting marker may be a particulate marker comprising a matrix material and the light-emitting polymer. The matrix material is preferably an inorganic matrix material, e.g. silica. According to these embodiments, the binding group may be bound, preferably covalently bound, to the matrix. In use, e.g. during flow cytometry, the light-emitting marker may be dispersed in a sample to be analysed.

Light-Emitting Polymer

The light-emitting polymer comprises a repeat unit of formula (I):

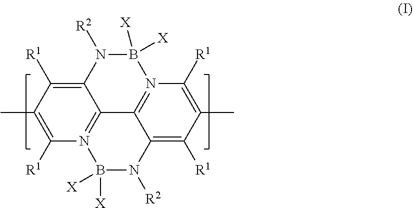

R¹ independently in each occurrence is H or a substituent.

R² independently in each occurrence is a substituent.

X independently in each occurrence is a substituent.

Preferably, R¹ is H.

Non-H groups R¹, where present, may each independently be selected from the group consisting of branched, linear or cyclic C₁₋₂₀ alkyl wherein one or more non-adjacent, non-terminal C atoms may be replaced with O, S, CO or COO and one or more H atoms may be replaced with F; and ionic substituents.

By “non-terminal C atom” of an alkyl group as used herein means a C atom other than the methyl group at the end of an n-alkyl chain or the methyl groups at the ends of a branched alkyl chain.

Preferably, R² independently in each occurrence is selected from the group consisting of branched, linear or cyclic C₁₋₂₀ alkyl; phenyl which is unsubstituted or substituted with one or more C₁₋₁₂ alkyl groups; and ionic substituents.

Preferably, X independently in each occurrence is selected from the group consisting of branched, linear or cyclic C₁₋₂₀ alkyl; phenyl which is unsubstituted or substituted with one or more substituents, e.g. one or more C₁₋₁₂ alkyl groups; and F.

Preferably, an ionic substituent as described herein has formula (II):

-(Sp)ₘ-(R³)ₙ (II)

wherein Sp is a spacer group; m is 0 or 1; R³ independently in each occurrence is an ionic group; n is 1 if m is 0 and n is at least 1, optionally 1, 2, 3 or 4, if m is 1.

Preferably, Sp is selected from:
C₁₋₂₀ alkylene or phenylene-C₁₋₂₀ alkylene wherein one or more non-adjacent C atoms may be replaced with O, S, N or C═O;
a C₆₋₂₀ arylene or 5-20 membered heteroarylene, more preferably phenylene, which, other than the one or more ionic groups R³, may be unsubstituted or substituted with one or more non-ionic substituents, optionally one or more non-ionic substituents R¹ as described above.

More preferably, Sp is selected from:
C₁₋₂₀ alkylene wherein one or more non-adjacent C atoms may be replaced with O, S or CO; and
a C₆₋₂₀ arylene or a 5-20 membered heteroarylene, even more preferably phenylene, which may be unsubstituted or substituted with one or more non-ionic substituents.

In a preferred embodiment, Sp is a C₆₋₂₀ arylene or 5-20 membered heteroarylene, more preferably phenylene, substituted with a group of formula (III):

—O(R⁴O)ᵥ—R⁵ (III)

wherein R⁴ in each occurrence is a C₁₋₁₀ alkylene group, optionally a C₁₋₅ alkylene group, wherein one or more non-adjacent, non-terminal C atoms of the alkylene group may be replaced with O, R⁵ is H or C₁₋₅ alkyl, and v is 0 or a positive integer, optionally 1-10. Preferably, v is at least 2. More preferably, v is 2 to 5. The value of v may be the same in all the polar groups of formula —O(R⁴O)ᵥ— R⁵. The value of v may differ between different groups of formula (II) of the same polymer.

Optionally, the group of formula (III) has formula —O(CH₂CH₂O)ᵥR⁵ wherein v is at least 1, optionally 1-10 and R⁵ is a C₁₋₅ alkyl group, preferably methyl. Preferably, v is at least 2. More preferably, v is 2 to 5, most preferably v is 3.

The ionic group R³ may be anionic or cationic.

Exemplary anionic group are —COO⁻, a sulfonate group; hydroxide; sulfate; phosphate; phosphinate; or phosphonate.

An exemplary cationic group is —N(R⁶)₃⁺ wherein R⁶ in each occurrence is H or C₁₋₁₂ hydrocarbyl. Preferably, each R⁶ is a C₁₋₁₂ hydrocarbyl.

A light-emitting polymer comprising cationic or anionic groups comprises counterions to balance the charge of these ionic groups. An anionic or cationic group and counterion may have the same valency, with a counterion balancing the charge of each anionic or cationic group. The anionic or cationic group may be monovalent or polyvalent. Preferably, the anionic and cationic groups are monovalent.

The light-emitting polymer may comprise a plurality of anionic or cationic polar groups wherein the charge of two or more anionic or cationic groups is balanced by a single counterion. Optionally, the polar groups comprise anionic or cationic groups comprising di- or trivalent counterions.

In the case of an anionic group, the cation counterion is optionally a metal cation, optionally Li⁺, Na⁺, K⁺, Cs⁺, preferably Cs⁺, or an organic cation, optionally ammonium, such as tetraalkylammonium, ethylmethyl imidazolium or pyridinium.

In the case of a cationic group, the anion counterion is optionally a halide; a sulfonate group, optionally mesylate or tosylate; hydroxide; carboxylate; sulfate; phosphate; phosphinate; phosphonate; or borate.

In some preferred embodiments, all repeat units are repeat units of formula (I).

In some preferred embodiments, the polymer is a copolymer comprising repeat units of formula (I) and one or more co-repeat units. Optionally, the copolymer comprises electron-accepting repeat units of formula (I) and electron-donating repeat units.

Preferably, the repeat units of a copolymer as described herein comprise or consist of a repeating structure of formula (III):

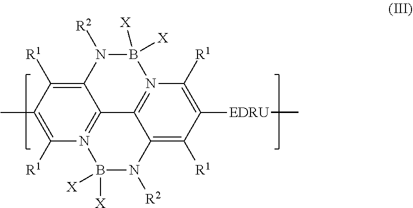

wherein EDRU is an electron-donating repeat unit.

A monomer for forming an electron-donating co-repeat unit as described herein may have a shallower LUMO level than a corresponding monomer for forming a repeat unit of formula (I). LUMO levels as described herein may be as measured by square wave voltammetry.

Preferably, the electron-donating co-repeat unit comprises at least one thiophene or furan which may be unfused or fused to one or more further rings.

Optionally, the polymer comprises an electron-donating repeat unit selected from formulae (IVa)-(IVp):

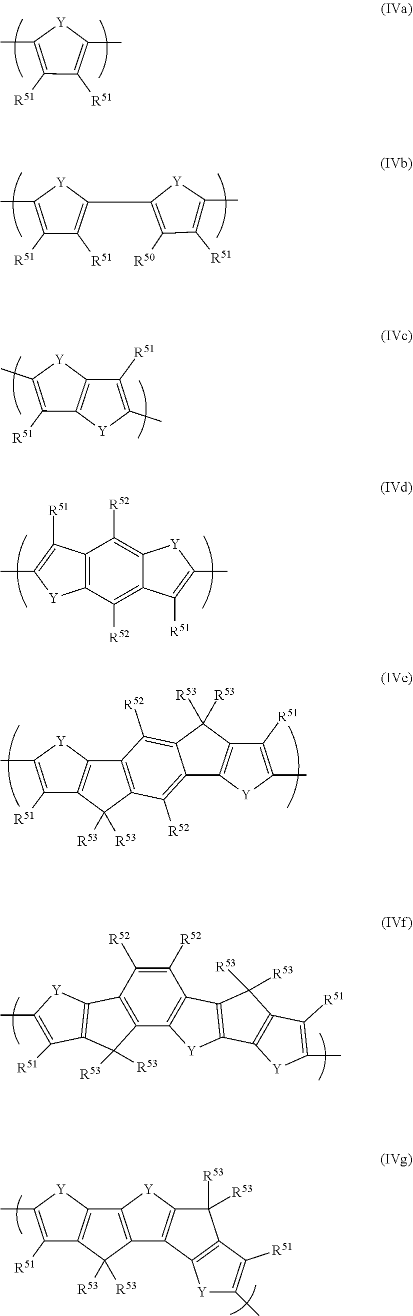

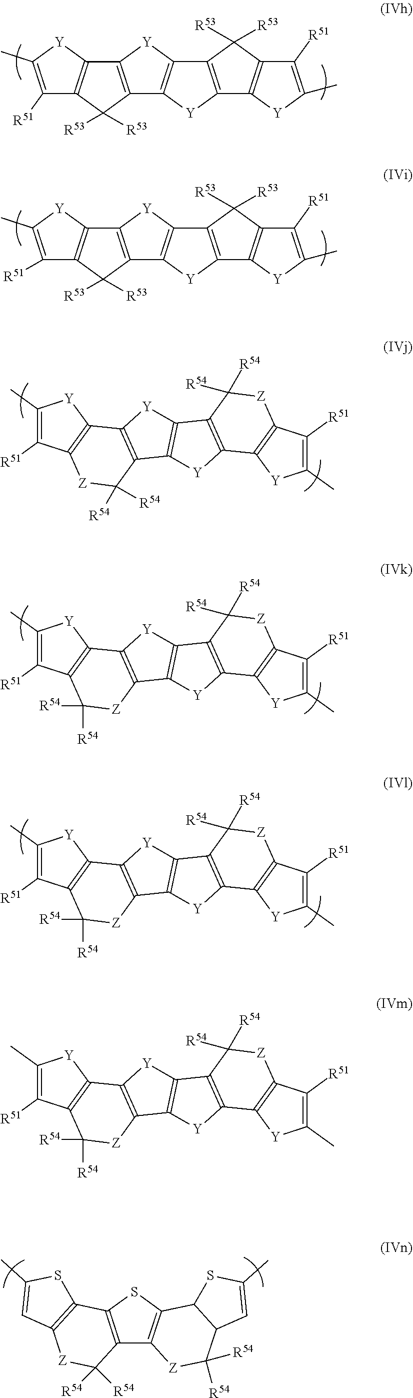

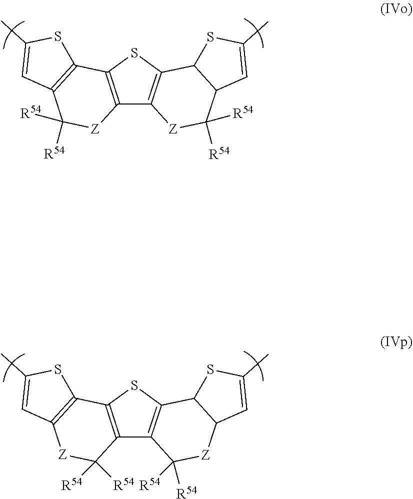

wherein Y in each occurrence is independently O or S; Z in each occurrence is O, S, NR⁵⁵, or C(R⁵⁴)₂; and R⁵⁰, R⁵¹, R⁵², R⁵³, R⁵⁴ and R⁵⁵ independently in each occurrence is H or a substituent and wherein R⁵⁰ groups may be linked to form a ring.

Optionally, R⁵⁰, R⁵¹ and R⁵² independently in each occurrence are selected from H; F; C₁₋₂₀ alkyl wherein one or more non-adjacent, non-terminal C atoms may be replaced with O, S, COO or CO and one or more H atoms of the alkyl may be replaced with F; an aromatic or heteroaromatic group Ar³ which is unsubstituted or substituted with one or more substituents; and an ionic substituent, preferably an ionic substituent of formula (II).

In some embodiments, Ar³ maybe an aromatic group, e.g. phenyl. Where present, substituents of Ar³ may be selected from R⁸, wherein R⁸ independently in each occurrence is selected from F, CN, NO₂ and C₁₋₂₀ alkyl wherein one or more non-adjacent, non-terminal C atoms may be replaced with O, S, COO or CO and one or more H atoms of the alkyl may be replaced with F.

In some preferred embodiments, each R⁵¹ is H.

Optionally, R⁵³ independently in each occurrence is selected from H; F; C₁₋₂₀ alkyl wherein one or more non-adjacent, non-terminal C atoms may be replaced with O, S, COO or CO and one or more H atoms of the alkyl may be replaced with F; an aromatic or heteroaromatic group Ar³ which is unsubstituted or substituted with one or more substituents; and an ionic substituent preferably an ionic substituent of formula (II).

Preferably, each R⁵⁴ is selected from the group consisting of:

H;

linear, branched or cyclic C₁₋₂₀ alkyl, wherein one or more non-adjacent, non-terminal C atoms may be replaced by O, S, NR⁷, CO or COO wherein R⁷ is a C₁₋₁₂ hydrocarbyl and one or more H atoms of the C₁₋₂₀ alkyl may be replaced with F;

a group of formula (Ak)u-(Ar⁴)v wherein Ak is a C₁₋₁₂ alkylene chain wherein one or more non-adjacent C atoms may be replaced with O, S, CO or COO; u is 0 or 1; Ar⁴ in each occurrence is independently an aromatic or heteroaromatic group, preferably a phenylene group, which is unsubstituted or substituted with one or more substituents; and v is at least 1, optionally 1, 2 or 3; and

an ionic group, preferably an ionic group of formula (II).

R⁵⁵ is optionally selected from H and a C₁₋₂₀ hydrocarbyl group. The C₁₋₂₀ hydrocarbyl group is optionally selected from C₁₋₂₀ alkyl and phenyl which is unsubstituted or substituted with one or more C₁₋₁₂ alkyl groups.

Optionally Ar⁴, where present, is unsubstituted or substituted with one or more substituents R⁸ as described above.

Preferably, each R⁵⁰ is a substituent. In a preferred embodiment, the R⁵⁰ groups are linked to form a group of formula —Z—C(R⁵⁴)₂— wherein Z is O, NR⁵⁵, or C(R⁵⁴)₂ and R⁵⁵ is a C₁₋₂₀ hydrocarbyl group, e.g. a group of formula (IIb-1), (IIb-2) or (IIb-3):

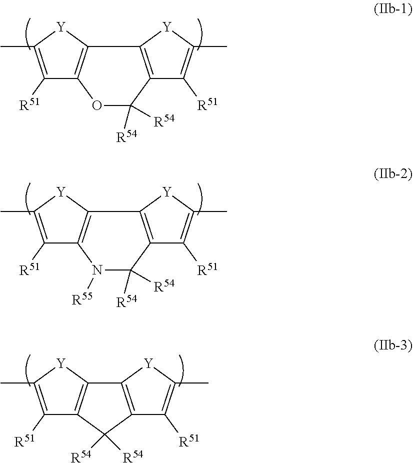

The polymer preferably has a solubility in water or a C₁₋₆ alcohol, preferably methanol, at 20° C. of at least 0.1 mg/ml.

To provide solubility in water, one or more of the repeat units of the polymer may be substituted with one or more polar, non-ionic substituents and/or ionic substituents.

In some embodiments, the repeat unit of formula (I) is substituted with an ionic substituent, e.g. a group of formula (II), and/or a polar, non-ionic substituent, e.g. a group of formula (III).

In some embodiments, the electron-donating repeat unit is substituted with an ionic substituent, e.g. a group of formula (II), and/or a polar, non-ionic substituent, e.g. a group of formula (III).

In some preferred embodiments, at least one substituent of the light-emitting polymer comprises is selected from —O(R⁴O)ᵥ—R⁵ and/or ionic groups, more preferably groups of formula —O(CH₂CH₂O)ᵥR⁵ and —COO⁻.

The polymer preferably has an absorption peak at a wavelength greater than 550 nm, preferably in the range of 550-750 nm.

The polymer preferably has an absorption peak at a wavelength greater than 550 nm having a full width at half maximum (FWHM) of no more than 100 nm.

Unless stated otherwise, emission spectra of light-emitting markers as described herein are as measured in methanol or water, using a Hamamatsu C9920-02 instrument having a set up wavelength 300 nm-950 nm; light source 150 W xenon light and bandwidth 10 nm or less (FWHM). Initially the system was calibrated with red (395 nm), green (375 nm) and blue (335 nm) glass standards. Two 5 ml long necked cuvettes (one filled with reference solvent i.e. water) and one filled with a sample of 1 mg/ml diluted 1 in 100 for a dissolved light-emitting marker or 1 mg/ml diluted ˜1 in 10 with water for a particulate light-emitting marker. The final concentration of the sample was altered to obtain a transmission data in the range 0.25-0.35. An average of 3 measurements for each sample is recorded.

Unless stated otherwise, absorption spectra of light-emitting materials as described herein are measured in methanol or water using a Cary 5000 UV-VIS-NIR Spectrometer. Measurements were taken from 175 nm to 3300 nm using a PbSmart NIR detector for extended photometric range with variable slit widths (down to 0.01 nm) for optimum control over data resolution. A baseline run with water in front and back 5 ml matched cuvettes (600 to 250 nm) following which the back cuvette reference remained as water and the front cuvette was changed to a sample of 1 mg/ml diluted 1 in 100 for a dissolved light-emitting marker or 1 mg/ml diluted ˜1 in 10 with water for a particulate light-emitting marker.

Conjugated light-emitting polymers as described herein may be formed by polymerising monomers comprising leaving groups that leave upon polymerisation of the monomers to form conjugated repeat units. Exemplary polymerization methods include, without limitation, Yamamoto polymerization as described in, for example, T. Yamamoto, “Electrically Conducting And Thermally Stable pi-Conjugated Poly(arylene)s Prepared by Organometallic Processes”, Progress in Polymer Science 1993, 17, 1153-1205, the contents of which are incorporated herein by reference, Suzuki polymerization as described in, for example, WO 00/53656, WO 2003/035796, and U.S. Pat. No. 5,777,070, the contents of which are incorporated herein by reference, Stille polymerization as described in, for example Macromolecules, 2017, 50, 8, 3171-3178 or direct arylation polymerisation as described in, for example Molecules, 2018, 23, 922.

Preferably, the polystyrene-equivalent number-average molecular weight (Mn) measured by gel permeation chromatography of the polymer is in the range of about 5×10³ to 1×10⁸, and preferably 1×10⁴ to 5×10⁶. The polystyrene-equivalent weight-average molecular weight (Mw) of the polymer may be 1×10³ to 1×10⁸, and preferably 1×10⁴ to 1×10⁷.

Polymers as described herein include, without limitation:

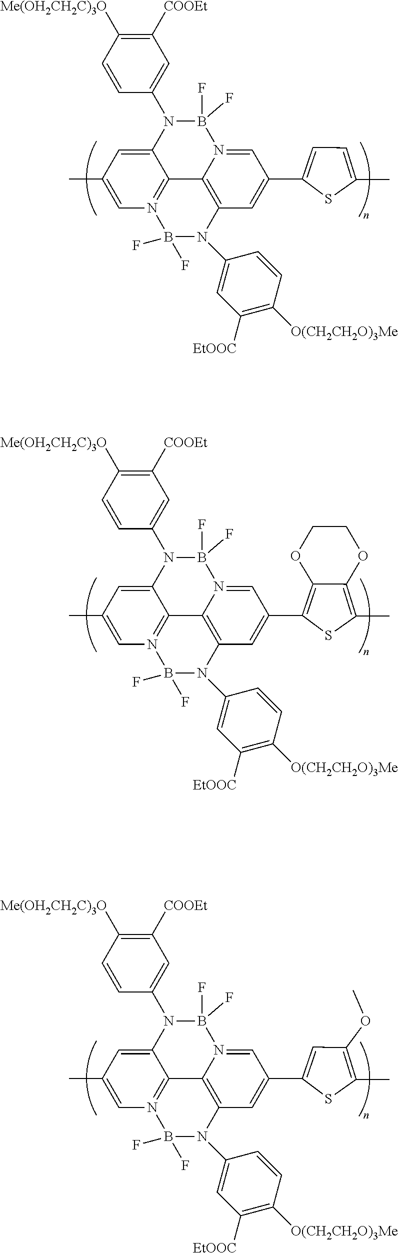

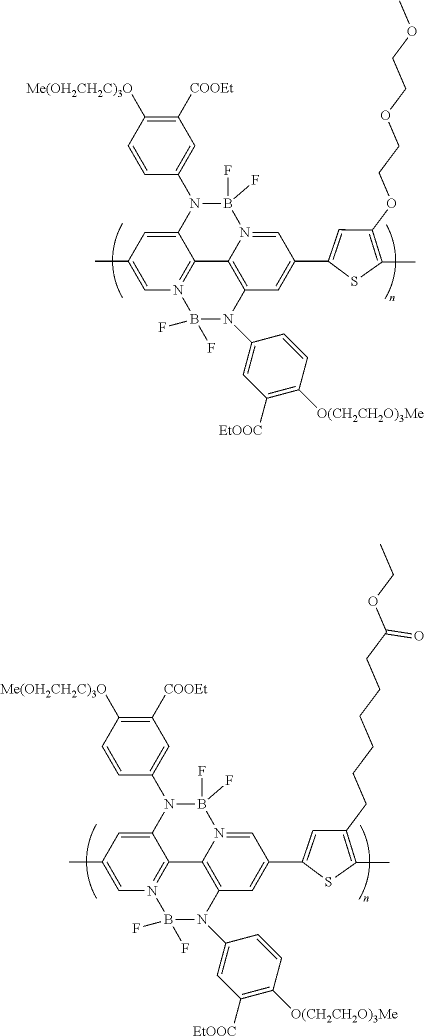

Light-Emitting Particle

The light-emitting marker may be in the form of a light-emitting particle comprising or consisting of the light-emitting material.

In some embodiments, formation of a light-emitting nanoparticle comprising or consisting of a polymer as described herein may include collapse of the light-emitting polymer.

In some embodiments, the particle may have a particulate core and, optionally, a shell wherein at least one of the core and shell contains the light-emitting material. Preferably, the light-emitting particle contains the light-emitting material and a matrix material. Matrix materials include, without limitation, inorganic matrix materials, optionally inorganic oxides, optionally silica. The matrix may at least partially isolate the light-emitting material from the surrounding environment. This may limit any effect that the external environment may have on the lifetime of the light-emitting material.

Polymer chains of a light-emitting polymer may extend across some or all of the thickness of the core and/or shell. Polymer chains may be contained within the core and/or shell or may protrude through the surface of the core and/or shell.

The light-emitting polymer may be mixed with the matrix material.

The light-emitting polymer may be bound, e.g. covalently bound, to the matrix material.

In some embodiments, the particle core may be formed by polymerisation of a silica monomer in the presence of the light-emitting polymer, for example as described in WO 2018/060722, the contents of which are incorporated herein by reference.

In some embodiments, the particle core comprises a core which comprises or consists of the light-emitting polymer and at least one shell surrounding the inner core. The at least one shell may be silica.

Optionally, at least 0.1 wt % of total weight of the particle core consists of the light-emitting polymer. Preferably at least 1, 10, 25 wt % of the total weight of the particle core consists of the light-emitting polymer.

Optionally at least 50 wt % of the total weight of the particle core consists of the matrix material. Preferably at least 60, 70, 80, 90, 95, 98, 99, 99.5, 99.9 wt % of the total weight of the particle core consists of the matrix material.

The particle core as described herein is the light-emitting particle without any surface groups, e.g. binding groups or solubilising groups, thereon.

In one embodiment of the present disclosure, at least 70 wt % of the total weight of the particle core consists of the light-emitting material or materials and silica. Preferably at least 80, 90, 95, 98, 99, 99.5, 99.9 wt % of the total weight of the particle core consists of the light-emitting material and silica. More preferably the particle core consists essentially of the light-emitting material and silica.

Preferably, the particles have a number average diameter of no more than 5000 nm, more preferably no more than 2500 nm, 1000 nm, 900 nm, 800 nm, 700 nm, 600 nm, 500 nm or 400 nm as measured by dynamic light scattering (DLS) using a Malvern Zetasizer Nano ZS. Preferably the particles have a number average diameter of between 5-5000 nm, optionally 10-1000 nm, preferably between 10-500 nm, most preferably between 10-100 nm as measured by a Malvern Zetasizer Nano ZS.

Light-emitting particles as described herein may be provided as a colloidal suspension comprising the particles suspended in a liquid. Preferably, the liquid is selected from water, C₁₋₁₀ alcohols and mixtures thereof. Preferably, the particles form a uniform (non-aggregated) colloid in the liquid. In some embodiments, each of the first, second and any further light-emitting markers are light-emitting particles dispersed in the liquid. In some embodiments, one or more of the light-emitting markers is in particle form dispersed in the liquid and one or more of the light-emitting markers is dissolved in the liquid.

The liquid may be a solution comprising salts dissolved therein, optionally a buffer solution.

In some embodiments, the particles may be stored in a powder form, optionally in a lyophilised or frozen form.

Functional Groups

The binding group of the light-emitting marker for binding to a target analyte may be attached to the light-emitting marker by attachment to a functional group of a precursor of the light-emitting marker. In some embodiments, the functional group is covalently bound to the light-emitting material. In some embodiments, the functional group is covalently bound to a matrix material of a precursor comprising the matrix material and the light-emitting material.

Optionally the functional group is selected from:

amine groups, optionally —NR⁹₂ wherein R⁹ in each occurrence is independently H or a substituent, preferably H or a C₁₋₅ alkyl, more preferably H;

carboxylic acid or a derivative thereof, for example an anhydride, acid chloride or ester, acid chloride, acid anhydride or amide group;

—OH; —SH; an alkene; an alkyne; and an azide; and

biotin or a biotin-protein conjugate.

The functional group may be reacted with a biomolecule to form a linking group linking the biomolecule to the rest of the light-emitting marker, the linking group being selected from esters, amides, urea, thiourea, Schiff bases, a primary amine (C—N) bond, a maleimide-thiol adduct or a triazole formed by the cycloaddition of an azide and an alkyne.

Exemplary binding group biomolecules for binding to a target analyte include, without limitation, DNA, RNA, peptides, carbohydrates, antibodies, antigens, enzymes, proteins, hormones and combinations thereof.

In the case where the functional group is biotin, it may be conjugated to a protein, e.g. avidin, streptavidin, neutravidin and recombinant variants thereof, and a biotinylated biomolecule may be conjugated to the protein to form the light-emitting marker.

The biotinylated biomolecule may comprise an antigen binding fragment, e.g. an antibody, which may be selected according to a target antigen.

In the case of a light-emitting particle, the functional group may be bound to a surface of the particle core, e.g. bound to a matrix material of the light-emitting particle core. Each functional group may be directly bound to the surface of a light-emitting particle core or may be spaced apart therefrom by one or more surface binding groups. The surface binding group may comprise polar groups. Optionally, the surface binding group comprises a polyether chain. By “polyether chain” as used herein is meant a chain having two or more ether oxygen atoms.

The surface of a light-emitting particle core may be reacted to form a group at the surface capable of attaching to a functional group. Optionally, a silica-containing particle is reacted with a siloxane.

Applications

Light-emitting markers as described herein may be used as luminescent probes for detecting or labelling a biomolecule or a cell. In some embodiments, the particles may be used as a luminescent probe in an immunoassay such as a lateral flow or solid state immunoassay. Optionally the particles are for use in fluorescence microscopy, flow cytometry, next generation sequencing, in-vivo imaging, or any other application where a light-emitting marker is brought into contact with a sample to be analysed. The analysis may be performed using time-resolved spectroscopy. The applications can medical, veterinary, agricultural or environmental applications whether involving patients (where applicable) or for research purposes.

In use the binding group of the light-emitting markers may bind to target biomolecules which include without limitation DNA, RNA, peptides, carbohydrates, antibodies, antigens, enzymes, proteins and hormones. The target biomolecule may or may not be a biomolecule, e.g. a protein, at a surface of a cell.

A sample to be analysed may brought into contact with the light-emitting marker, for example the light-emitting marker dissolved in a solution or a particulate light-emitting marker in a colloidal suspension.

In some embodiments, the sample is analysed by flow cytometry. In flow cytometry, the light-emitting marker or markers are irradiated by at least one wavelength of light, optionally two or more different wavelengths, e.g. one or more wavelengths including at least one of about 355, 405, 488, 530, 561 and 640 nm, each of which may be ±10 nm. Light emitted by the light-emitting marker(s) may be collected by one or more detectors. To provide a background signal for calculation of a staining index, measurement may be made of a light-emitting marker mixed with cells which do not bind to the light-emitting marker.

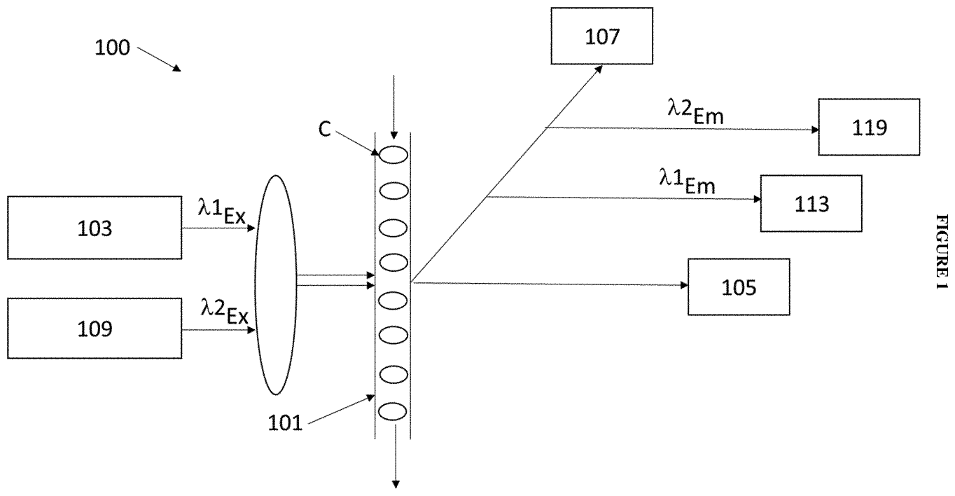

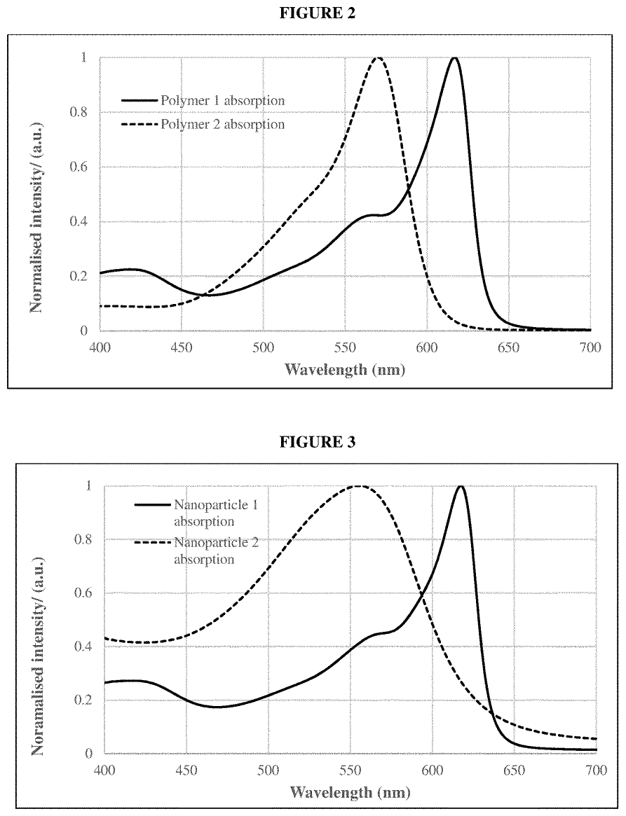

FIG. 1 schematically illustrates flow cytometer 100 which may be used in some embodiments of the flow cytometry methods of the present disclosure.

The flow cytometer comprises a flow channel 101 through which cells may pass in a single file; a first light source 103, e.g. a laser, configured to irradiate the flow channel with light of a first excitation wavelength λ1Ex; a forward scatter detector 105; a side scatter detector 107; and a first photodetector 113 configured to detect light of a first emission wavelength λ1Em emitted from a first light-emitting marker bound to a cell upon excitation by the first excitation wavelength λ1Ex, wherein the first light-emitting marker comprises a light-emitting polymer as described herein.

The apparatus may further comprise at least one further light source 105, e.g. a laser, configured to irradiate the flow channel with light of a second excitation wavelength λ2Ex and a photodetector configured to detect light of a second emission wavelength λ2Em emitted from at least one further light-emitting marker bound to a cell upon excitation by the second excitation wavelength λ2Ex.

The one or more further light-emitting markers contain a light-emitting material which is different from the light-emitting polymer of the first light-emitting marker. λ1Ex and λ2Ex are different. λ1Em and λ2Em are different.

For simplicity, FIG. 1 illustrates a flow cytometer having only two light sources and only two photodetectors however it will be understood that in other embodiments the flow cytometer may have more than two light sources and/or more than two photodetectors. A single light source may be configured to excite a single light-emitting marker of a plurality of light-emitting markers present in a sample being analysed or may be configured to excite a plurality of different light-emitting markers.

In some embodiments, a sample to be analysed contains a plurality of light-emitting markers including a light-emitting marker as described herein. Preferably, a light-emitting marker comprising a polymer comprising a repeat unit of formula (I) as described herein has a full width at half maximum (FWHM) which is separated by at least 100 nm from the FWHM of the light-emitting materials of the one or more further light-emitting markers.

Signals received by the forward scatter detector, side scatter detector and photodetectors may be transmitted by wired or wireless transmission to a signal processor (not shown).

A method of sequencing nucleic acids may comprise:

contacting a primed template nucleic acid molecule with a polymerase and a test nucleotide;

incorporating the test nucleotide into a primed strand of the primed template only if it comprises a base complementary to the next base of the template strand;

irradiating the primed strand; and

determining from luminance of the primed strand if the test nucleotide has been incorporated into the primed strand,

wherein the test nucleotide of the irradiated primed strand is bound to a light-emitting polymer or light-emitting particle as described anywhere herein.

### Examples

Monomers

Monomer Example 1 was formed according to Scheme 1. Intermediate 5 of Scheme 1 was formed according to Scheme 2.

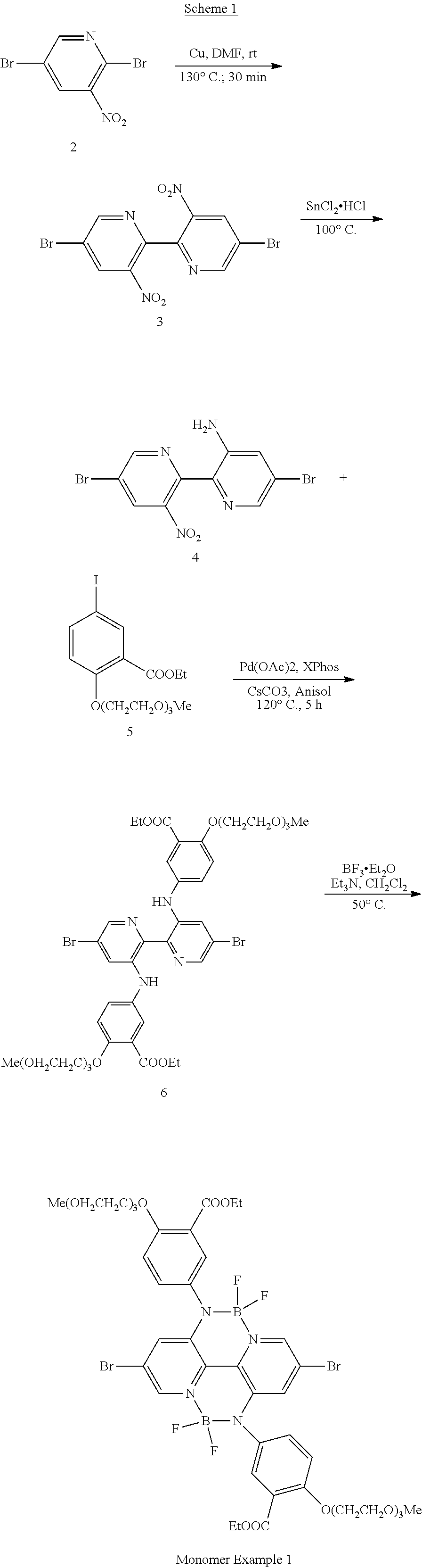

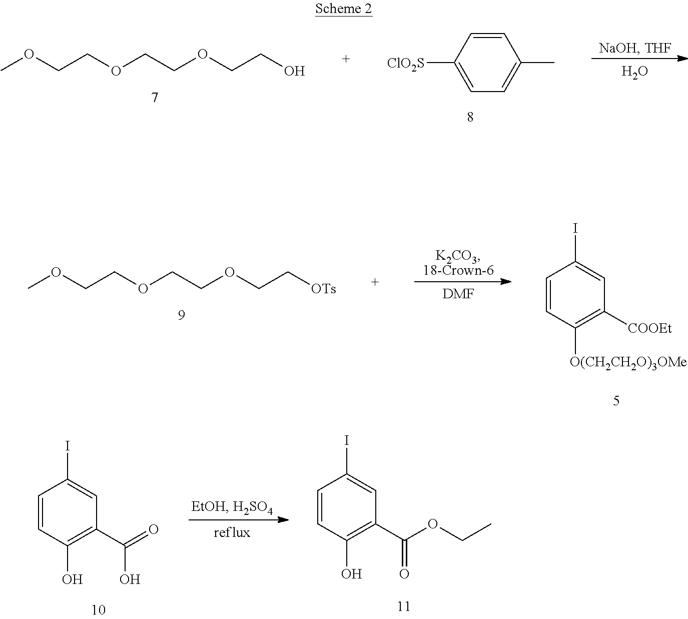

Intermediate 4 is known and was synthesized according to the procedure described in Angew. Chem. Int. Ed., 2016, 55, 1436, the contents of which are incorporated herein by reference.

Synthesis of intermediate 9: In a 3 L three necked RB flask with mechanical stirring, Triethylene glycol monomethyl ether (100 g, 609 mmol) and sodium hydroxide (31.4 g, 786 mmol) were taken in THF-water mixture (350 mL-50 mL). 4-toluenesulfonyl chloride (107 g, 622 mmol) was slowly added at 0° C. under nitrogen atmosphere. (Observed formation of a dense white gas. The reaction mixture was slowly allowed to room temperature and stirred for overnight (˜18 h). After completion of the reaction, the mixture was carefully poured into water (500 mL), extracted with dichloromethane (1 L×2 times). The combined organic phase washed with 3M HCl (250 mL), sodium bicarbonate (500 mL), and water (500 mL), dried over sodium sulfate and concentrated. The crude product was passed through a short column chromatography to get 120 g of intermediate 9. The structure was confirmed spectroscopically.

¹H-NMR (400 MHz, CDCl₃): δ [ppm] 2.47 (s, 3H), 3.41 (s, 3H), 3.54-3.56 (m, 2H), 3.61-3.65 (m, 6H), 3.69-3.72 (m, 2H), 4.17-4.20 (m, 2H), 7.82 (d, J=8.4 Hz, 2H), 7.36 (d, J=8.4 Hz, 2H).

Synthesis of intermediate 11: In a 3 L three necked round-bottomed flask, equipped with overhead stirrer, 2-hydroxy-5-iodobenzoic acid 10 (95 g, 359 mmol) was taken in Ethanol (1000 mL) at room temperature (25° C.). Thionyl chloride (51.1 g, 430 mmol, 35 mL) was added and then reaction mass was slowly heated to 90° C. and stirred for 18 h. The mixture was cooled to room temperature and concentrated in rotary evaporator under vacuum to remove ethanol. The residue was dissolved in ethyl acetate (1 L) and washed with saturated sodium bicarbonate solution (500 mL), water (1 L), saturated brine solution (500 mL), dried over sodium sulphate and concentrated to get 96 g of crude product as off white solid. It was purified by column chromatography using silica gel (230-400 mesh) and eluted with 5% dichloromethane in hexane to obtain 73 g of intermediate 11 with 98.39% pure white solid.

¹H-NMR (400 MHz, CDCl₃): δ [ppm] 1.45 (t, J=7.2 Hz, 6H), 4.43 (q, J=7.2 Hz, 4H), 6.79 (d, J=8.8 Hz, 1H), 7.71 (dd, J=2.8 Hz, 8.8 Hz, 1H), 8.15 (d, J=2.0 Hz, 1H), 10.84 (s, 1H).

Synthesis of intermediate 5: In 3 L three necked round bottom flask, equipped with overhead stirrer condenser, a mixture of ethyl 2-hydroxy-5-iodobenzoate (11) (71 g, 243 mmol), Tosyl intermediate (9) (92.5 g, 291 mmol), 18-Crown 6 (12.8 g, 48.6 mmol) and potassium carbonate (83.8 g, 607 mmol) in N,N,-Dimethylformamide (350 mL) was heated to 105° C. for 5 h. Reaction progress was monitored by LCMS. After 5 h LCMS showed 95.81% of desire product formation. The reaction mixture was cooled to room temperature, carefully added to water (1 L) and extracted with ethyl acetate (1000 mL×3). The combined organic layer was washed with aqueous brine solution (1 L). Dried over sodium sulphate and concentrated to get 130 g of crude product. The crude product was purified by silica column chromatography (230-400 mesh silica) and eluted with 15% ethyl acetate in hexane, and desired product fractions were concentrated to get 100 g of intermediate 4 with 98% LCMS purity. ¹H-NMR (400 MHz, CDCl₃): δ [ppm] 1.39 (t, J=7.2 Hz, 3H), 3.41 (s, 3H), 3.56-3.58 (m, 2H), 3.68-3.73 (m, 4H), 3.76-3.79 (m, 2H), 3.79-3.81 (m, 2H), 3.89-3.91 (m, 2H), 4.36 (q, J=7.2 Hz, 2H), 6.79 (d, J=8.8 Hz, 1H), 7.71 (dd, J=2.4 Hz, 8.8 Hz, 1H), 8.05 (d, J=2.4 Hz, 1H).

Synthesis of intermediate 6: In a 1 L round bottom flask was taken Anisole (300 mL) and purged with nitrogen for 30 min. Then added Xantphos (1.76 g, 3.05 mmol) and Palladium acetate (0.685 g, 3.05 mmol) with nitrogen purging and continued nitrogen purging for another 15 min, then added 5,5′-dibromo-[2,2′-bipyridine]-3,3′-diamine (21.0 g, 61.04 mmol), ethyl 5-iodo-2-(2-(2-(2-methoxyethoxy)ethoxy)ethoxy)benzoate (56.15 g 128.19 mmol) and Cesium carbonate (49.72 g, 152.65 mmol). Then the reaction was stirred at 120° C. for 5 h. The reaction progress was monitored by LCMS. After completion of the reaction, it was cooled to room temperature, then diluted with DCM and filtered through Florisil bed. The filtrate was washed with water and the organic layer was dried over sodium sulphate and concentrated to get 50 g of crude intermediate 5. The crude was purified by gravity column chromatography (60-120 mesh silica), desired product eluted at 100% ethyl acetate to obtain 30 g of product with 96.4% LCMS purity. It was further recrystallized using ethyl acetate:hexane (1:5) at 50° C. to get 27.0 g of product with 97.62% LCMS purity. ¹H-NMR (400 MHz, CDCl₃): δ [ppm] 1.41 (t, J=7.2 Hz, 6H), 3.41 (s, 6H), 3.57-3.60 (m, 4H), 3.67-3.73 (m, 8H), 3.79-3.83 (m, 4H), 3.94 (t, J=5.2 Hz, 4H), 4.26 (t, J=5.2 Hz, 4H), 4.38 (q, J=7.2 Hz, 4H), 7.07 (d, J=8.8 Hz, 2H), 7.36 (dd, J=2.8 Hz, 8.8 Hz, 2H), 7.49 (d, J=2.0 Hz, 2H), 7.67 (d, J=2.8 Hz, 2H), 8.04 (d, J=2.0 Hz, 2H), 11.29 (s, 1H).

Synthesis of Monomer 1: In a 2 L round bottom flask Intermediate 5 (27 g, 28.0 mmol) and trimethylamine (32.4 mL) were taken in DCM (1080 mL), cooled the reaction mixture to 0° C. and added Boron trifluoride diethyl etherate (172.74 mL, 1400 mmol) at 0° C. The reaction mixture was stirred at 50° C. for 25 h and reaction progress was monitored by LCMS, crude LCMS analysis showed 64.88% of desired product formation and 30% of mono coupled product. Reaction was cooled to room temperature, quenched with water and extracted with DCM, the organic layer was dried over sodium sulphate and concentrated to get 52 g of crude product. The crude product was again treated with BF₃.Et₂O. The crude product (52.0 g), Trimethylamine (61.5 mL), were taken in DCM (2.2 L), cooled the reaction mixture to 0° C. and Boron trifluoride etherate (133.11 mL, 1078 mmol) was added at 0° C. The reaction mixture was stirred at 50° C. for 4 days and progress was monitored by LCMS which showed 90% of desired product formation and 2% of mono coupled product. The mixture was cooled to room temperature, quenched with water and extracted with DCM and organic layer was dried over sodium sulphate, concentrated to get 60 g of crude product.

Purification:

The crude product was taken for purification using grace column chromatography and further recrystallized using Ethyl acetate: hexane (1:1, 5 Volume) at 65° C. to obtain 13 g of 98.53% pure product with 0.95% early eluting impurity by LCMS.

2 g of the product was taken for reverse phase column chromatography purification utilizing Grace Instrument using C18 column. The desired product was eluted at 60 to 70% acetonitrile: water solvent system to get 1.2 g of pure product which was further recrystallized with Ethyl acetate: Hexane (1:1) to get 1 g product with 99.6% purity by LCMS.

¹H-NMR (400 MHz, CDCl₃): δ [ppm] 1.39 (t, J=7.2 Hz, 6H), 3.41 (s, 6H), 3.57-3.60 (m, 4H), 3.68-3.73 (m, 8H), 3.79-3.83 (m, 4H), 3.97 (t, J=5.2 Hz, 4H), 4.31 (t, J=5.2 Hz, 4H), 4.36 (q, J=7.2 Hz, 4H), 7.18 (d, J=9.2 Hz, 2H), 7.43 (dd, J=2.0 Hz, 8.8 Hz, 2H), 7.46 (d, J=2.0 Hz, 2H), 7.70 (d, J=2.4 Hz, 2H), 8.31 (d, J=2.0 Hz, 2H).

Polymers

The polymers 1 and 2 were synthesized following the procedure described in Macromolecules, 2017, 50, 8, 3171-3178 and EP1344788A1 respectively followed by hydrolysis of ester groups R′2 into cesium carboxylate groups R2 as disclosed in WO 2012/133229.

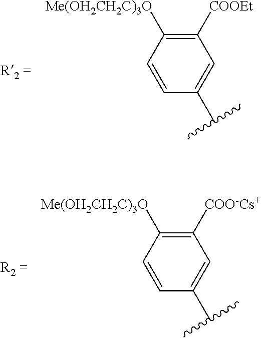

TABLE 1

| | Polymer structure | Physical stale/colour | λ_{abs}/nm | FWHM/nm |
|---|---|---|---|---|
| Polymer 1 | 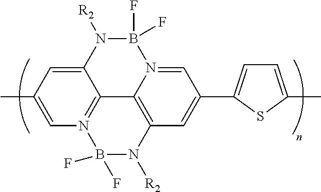 | Solid/dark purple | 618 | 39 |
| Polymer 2 | 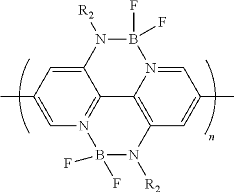 | Solid/dark pink | 572 | 60 |

Solution UV-VIS measurements: A Cary 5000 UV-VIS-NIR Spectrometer was used to measure solution UV-VIS of samples in methanol for polymer and in water for nanoparticles. Measurement done from 175 nm to 3300 nm using a PbSmart NIR detector for extended photometric range with variable slit widths (down to 0.01 nm) for optimum control over data resolution. Baseline run with solvent (methanol or water) in both 5 ml matched cuvettes (600 to 250 nm). Back cuvette reference remains as methanol or water and the front cuvette changed to a diluted sample of polymer nominally 1 mg/ml diluted 1 in 100 with methanol or the nanoparticle nominally 1 mg/ml diluted ˜1 in 10 with water. Table 1 shows that the light emitting polymer 1 and polymer 2 has an absorption peak of greater than 550 nm in methanol and full width half maxima (FWHM) is less than 100 nm.

Nanoparticles

Silica LEP nanoparticles were prepared as described in WO 2018/060722, the contents of which are incorporated herein by reference. Nanoparticle 1 and nanoparticle 2 contain Polymer 1 and Polymer 2 respectively and their absorption peaks are given in Table 2.

TABLE 2

| | Solvent | λ_{abs}/nm |
|---|---|---|
| Nanoparticle 1 | water | 619 |
| Nanoparticle 2 | water | 558 |

1. A method of identifying a target analyte in a sample, the method comprising irradiating the sample to which has been added a first light-emitting marker configured to bind to the target analyte; and detecting emission from the sample at a first emission wavelength of the light-emitting marker, wherein the light-emitting marker comprises a light-emitting polymer comprising a repeat unit of formula (I): 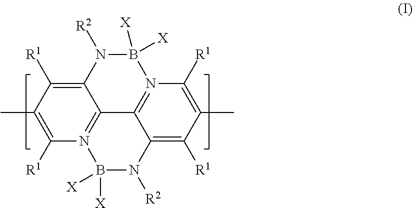
wherein R¹ independently in each occurrence is H or a substituent; R² independently in each occurrence is H or a substituent; and X in each occurrence is independently a substituent.

2. The method according to claim 1 wherein the polymer is a homopolymer.

3. The method according to claim 1 wherein the polymer is a copolymer comprising an electron-accepting repeat unit of formula (I) and an electron-donating repeat unit.

4. The method according to claim 3 wherein the electron donating repeat unit is selected from formulae (IVa)-(IVp): 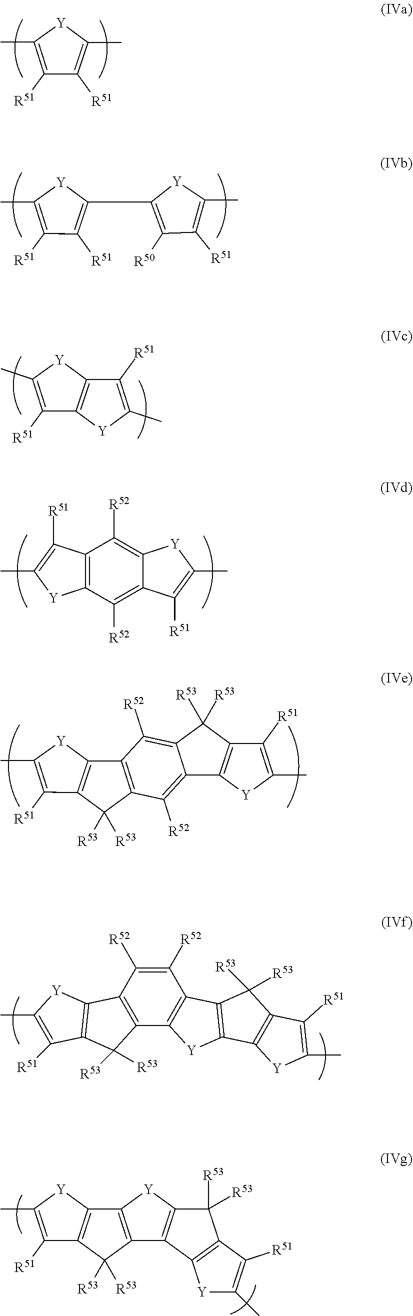 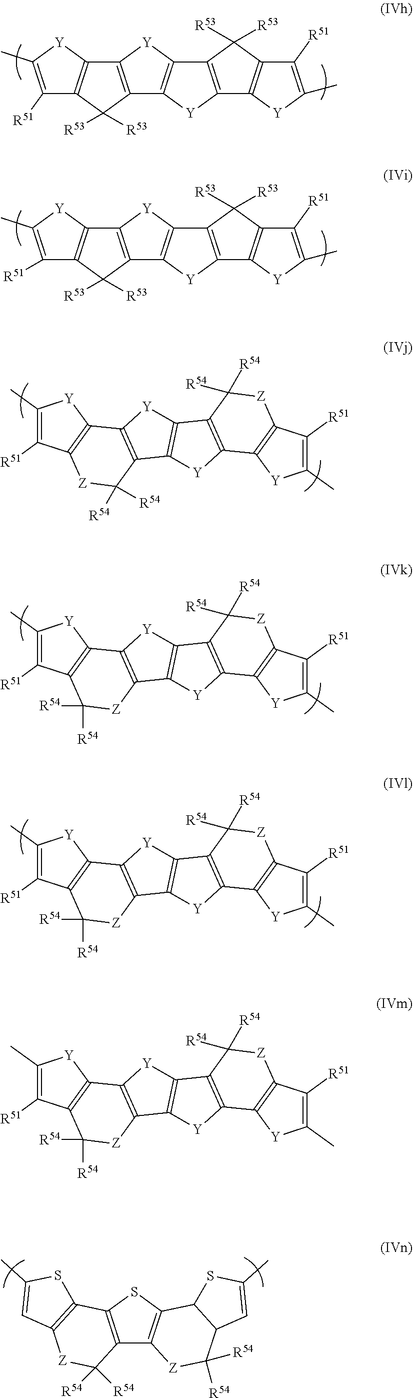
wherein Y in each occurrence is independently O or S; Z in each occurrence is O, NR⁵⁵, or C(R⁵⁴)₂; and R⁵⁰, R⁵¹, R⁵², R⁵³, R⁵⁴ and R⁵⁵ independently in each occurrence is H or a substituent and wherein R⁵⁰ groups may be linked to form a ring.

5. The method according to claim 1 wherein the light-emitting marker comprises a biomolecule configured to bind to the target analyte.

6. The method according to claim 1 wherein the polymer has a solubility in water or a C₁₋₆ alcohol of at least 0.1 mg/ml.

7. The method according to claim 1 wherein at least one of the repeat units of the light-emitting polymer is substituted with at least one ionic substituent.

8. The method according to claim 1 wherein at least one of the repeat units of the light-emitting polymer is substituted with at least one group of formula (III):
—O(R⁴O)ᵥ—R⁵ (III)
wherein R⁴ in each occurrence is a C₁₋₁₀ alkylene group wherein one or more non-adjacent, non-terminal C atoms of the alkylene group may be replaced with O, R⁵ is H or C₁₋₅ alkyl, and v is 0 or a positive integer.

9. The method according to claim 1 wherein the light-emitting polymer has an absorption peak of greater than 550 nm.

10. The method according to claim 1 wherein the light-emitting marker is a particulate marker.

11. (canceled)

12. (canceled)

13. The method according to claim 1 wherein the light-emitting marker is dissolved in the sample.

14. The method according to claim 1 wherein the sample comprises one or more additional light-emitting markers different from the first light-emitting marker.

15. The method according to claim 14 wherein each of the one or more additional light-emitting markers comprises an additional light-emitting material which emits light having an emission peak which is different from that of the light-emitting polymer.

16. (canceled)

17. (canceled)

18. The method according to claim 1 wherein the method is a flow cytometry method and the target analyte is a target cell.

19. A light-emitting marker precursor comprising a light-emitting polymer and a functional group wherein the light-emitting polymer comprises a repeat unit of formula (I): 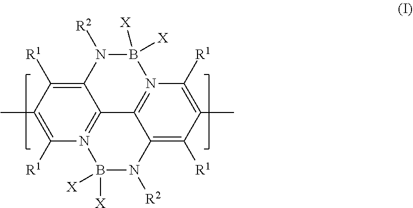
wherein R¹ independently in each occurrence is H or a substituent; R² independently in each occurrence is H or a substituent; and X in each occurrence is independently a substituent.

20. (canceled)

21. (canceled)

22. A formulation comprising the light-emitting marker precursor according to claim 19 dissolved or dispersed in one or more solvents.

23. A light-emitting marker comprising a light-emitting polymer and a binding group comprising a biomolecule wherein the light-emitting polymer comprises a repeat unit of formula (I): 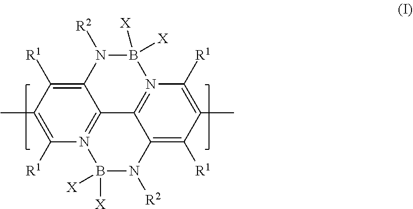
wherein R¹ independently in each occurrence is H or a substituent; R² independently in each occurrence is H or a substituent; and X in each occurrence is independently a substituent.

24. (canceled)

25. (canceled)

26. A light-emitting particle comprising a light-emitting polymer comprising a repeat unit of formula (I): 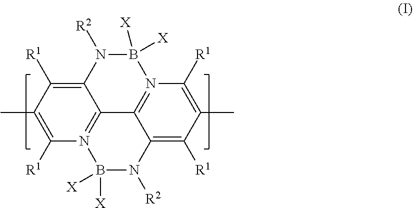
wherein R¹ independently in each occurrence is H or a substituent; R² independently in each occurrence is H or a substituent; and X in each occurrence is independently a substituent.

27-32. (canceled)

33. A formulation comprising light-emitting particles to claim 26 dispersed in one or more solvents.

34. A method of forming light-emitting particles according to claim 26 wherein a monomer for forming the silica is polymerised in the presence of the light-emitting material.
